(19) United States
(12) Patent Application Publication
(10) Pub. No.: US 2026/0096972 A1
(43) Pub. Date: Apr. 9, 2026

(54) Compositions Containing a UV-Absorbing System Including Physical UV-Attenuating Material and Fatty Acid Ester of Polyol

(71) Applicant: L'OREAL, PARIS (FR)

(72) Inventors: FRÉDÉRIC SIMONET, Chevilly-Larue (FR); AMÉLIE FRIOT, Chevilly-Larue (FR)

(73) Assignee: L'OREAL, PARIS (FR)

(21) Appl. No.: 18/906,316

(22) Filed: Oct. 4, 2024

(51) Int. Cl. A61K 8/29; A61K 8/06; A61K 8/27; A61K 8/37; A61Q 17/04

(52) U.S. Cl. CPC A61K 8/29; A61K 8/064; A61K 8/27; A61K 8/375; A61Q 17/04; A61K 2800/413; A61K 2800/621; A61K 2800/623; A61K 2800/651

(57) ABSTRACT

The present disclosure relates to compositions including a UV absorbing system (component) including at least one physical ultraviolet (UV)-attenuating material and a surfactant system (component) comprising at least one fatty acid ester of polyol, as well as to methods of making and using such compositions.

### FILED OF THE INVENTION

The present disclosure relates to compositions comprising a UV absorbing system (component) comprising at least one physical ultraviolet (UV)-attenuating material, and a surfactant system (component) comprising at least one fatty acid ester of polyol, as well as to methods of making and using such compositions.

### DISCUSSION OF BACKGROUND

Radiation of wavelengths between 290 nm and 400 nm allows tanning of the human epidermis, while radiation of wavelengths between 290 and 320 nm, called UVB rays, hinders the development of a natural tan. Exposure is also likely to lead to a detrimental change in the biomechanical properties of the epidermis, resulting in the appearance of wrinkles leading to premature aging of the skin (i.e., photoaging).

UVA rays with wavelengths between 320 and 400 nm penetrate deeper into the skin than UVB rays. UVA rays cause immediate and persistent browning of the skin. Daily exposure to UVA rays, even for a short time, under normal conditions can damage collagen fibers and elastin, resulting in a change in the microrelief of the skin, the appearance of wrinkles and uneven pigmentation (spots, lack of uniformity of complexion).

Many studies show the need for effective protection against UVA and UVB to prevent sunburn, photoaging, and the like.

In order to obtain a high protection product, it is generally necessary to combine a large number of sunscreens and/or a high amount of UV filters to achieve high levels of filtering efficiency.

However, high levels of UV filters do not lend themselves to an easy elaboration of stable compositions with a pleasant texture. It is possible to achieve high UV protection using one or more organic UV filters; however, organic UV filters are often very greasy and unpleasant when applied to the skin, especially when used at high levels in a sunscreen formulation required for high UV protection efficacy.

Mineral UV filters such as titanium dioxide or zinc oxide are alternative UV filter choices to organic UV filters which can bring more pleasant textures; however, when one or more mineral UV filters are used, the resulting sunscreen formulation is far too whitening (also referred to as “white cast”) when applied to the skin. When mineral UV filters are used at lower levels, it may be possible to produce sunscreen formulations with little to no whitening when applied to skin; however, such formulations containing low levels of mineral UV filters are not able to yield desired high levels of UV protection.

Also, combining organic UV filter and mineral UV filter in a single composition can be difficult, particularly doing so in a manner which results in a non-greasy, pleasant, non-whitening, high UV protection sunscreen formulation, especially considering the need to balance UVA and UVB protection efficacy.

Finally, consumers like fluid sunscreen compositions such as sprayable sunscreen compositions because they are generally easy to apply and spread during application. But it can be difficult to provide broad spectrum protection against UV rays and/or high SPF values to a stable, fluid emulsion composition containing mineral UV filters because the amount of mineral UV filter necessary to provide such broad spectrum and/or high SPF protection against UV rays is such that it can cause the fluid emulsion composition to be unstable and/or have poor application properties, making formulation of an acceptable composition problematic.

There remains a need in the art for improved fluid sunscreen compositions which possess high UV protection and balanced UVA and UVB protection, which are stable, not greasy, are pleasant to apply, and which are not whitening when applied to the skin.

Accordingly, one aspect of the present disclosure is a stable, fluid composition which is not greasy, which is non-whitening when applied to the skin, and which has high UV protection.

### SUMMARY OF THE INVENTION

The present disclosure relates to compositions comprising a UV absorbing system comprising at least one physical ultraviolet (UV)-attenuating material, and a surfactant system (component) comprising at least one fatty acid ester of polyol. Preferably, the compositions are fluid. Preferably, the compositions provide broad spectrum protection against UV rays. Preferably, the compositions provide SPF protection of at least 25, preferably at least 30. Preferably, the compositions are in the form of an emulsion, most preferably a water-in-oil emulsion.

The present disclosure also relates to methods of treating, caring for, protecting, enhancing the appearance of, and/or making up a keratinous material comprising applying compositions of the present disclosure to a keratinous material in an amount sufficient to treat, care for, enhance the appearance of, and/or make up the keratinous material.

The present disclosure also relates to methods of making compositions comprising at least one physical ultraviolet (UV)-attenuating material by combining at least one physical ultraviolet (UV)-attenuating material and at least one fatty acid ester of polyol during formation of the compositions. Preferably, the compositions are fluid. Preferably, the compositions are non-greasy and/or non-whitening (that is, the compositions do not provide a white cast) upon application. Preferably, the compositions provide broad spectrum protection against UV rays. Preferably, the compositions provide SPF protection of at least 25, preferably at least 30. Preferably, the compositions are in the form of an emulsion, most preferably a water-in-oil emulsion.

It is to be understood that both the foregoing general description and the following detailed description are exemplary and explanatory only, and are not restrictive of the disclosure.

### DETAILED DESCRIPTION OF THE INVENTION

In the following description and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5 includes specifically the integers within the range 1, 2, 3, 4 and 5, as well as subranges such as and 2-5, 3-5, 2-3, 2-4, 1-4, etc., as well as all fractional numbers within the range such as 1.2, 2.3, 3.4, etc., and subranges including such fractional numbers such as 1.5-3.8, 2-4.3, 4.2-4.9, etc.

“Fluid composition” as used herein refers a composition which is not solid at ambient temperature (25° C.) and for which it is possible to measure a viscosity. The term “fluid” is understood to mean a liquid composition which flows under its own weight at ambient temperature (25° C.) and atmospheric pressure. Advantageously, a fluid composition according to the present disclosure has a viscosity at 25° C. of less than 2000 mPa·s. According to preferred embodiments, the viscosity at 25° C. is less than 1000 mPa·s, preferably less than 500 mPa·s, and preferably less than 400 mPa·s.

“Film former”, “film-forming polymer” or “film-forming agent” as used herein means a polymer or resin which is capable of leaving a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as hydrogen atoms or chlorine atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the disclosure provided that these amounts do not materially affect at least one, preferably most, of the advantageous properties of the compositions of the disclosure. Thus, for example, “free of oil” means that an effective amount (that is, more than trace amounts) of oil(s) is/are omitted from the composition (that is, about 0% by weight), “substantially free of oil” means that oil(s) is/are present in amounts not greater than 0.1% by weight, and “devoid of oil” means that oil(s) is/are present in amounts not greater than 0.25% by weight, based on the total weight of the composition. The same nomenclature applies for all other ingredients identified throughout the application and in this paragraph such as, for example, specific UV filters and/or surfactants (compositions of the disclosure which are “free of oxybenzone and/or octinoxate,” “substantially free of oxybenzone and/or octinoxate,” and “devoid of oxybenzone and/or octinoxate,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes organic UV filters such as avobenzone, octocrylene, benzophenones, benzotriazoles and merocyanines, as well as physical ultraviolet (UV)-attenuating materials.

“Whitening” or “white cast” as used herein refers to the visually white appearance of keratinous substance after a composition has been applied to the keratinous material as compared to the appearance of the keratinous material prior to application of the composition. A composition is “non-whitening” if it provides minimal or no visually white appearance (preferably no visually white appearance) to keratinous material upon application.

“Anhydrous” as it is used herein means that compositions of the disclosure contain less than 3% water, meaning that the compositions can also contain less than 2% water, and less than 1% water, as well as being “free of water,” “substantially free of water,” and “devoid of water” as defined above.

A “UV absorbing system essentially containing at least one physical ultraviolet (UV)-attenuating material” as it is used herein means that compositions of the disclosure contain less than 3% UV filters other than physical ultraviolet (UV)-attenuating material(s), in particular less than 3% of UV organic filters, meaning that within this definition as subcategories are compositions containing less than 2% UV filters other than physical ultraviolet (UV)-attenuating material(s), in particular less than 2% of UV organic filters, and less than 1% UV filters other than physical ultraviolet (UV)-attenuating material(s), in particular less than 1% of UV organic filters, as well as being “free of UV filters other than physical ultraviolet (UV)-attenuating material,” “substantially free of UV filters other than physical ultraviolet (UV)-attenuating material,” and “devoid of UV filters other than physical ultraviolet (UV)-attenuating material” as defined above.

“System” and “component” are used interchangeably in this application.

“Primary Particle” as used in connection with the description of physical ultraviolet (UV)-attenuating material herein means inorganic or organic particles (structures) which can be held together via molecular or atomic bonding to form a physical ultraviolet (UV)-attenuating material.

“Primary particle size” means the size of an unaggregated primary particle in a physical ultraviolet (UV)-attenuating material.

“Passivated” as used in connection with the description of physical ultraviolet (UV)-attenuating material herein refers to a material which has been treated in a way such that the potential to release ionic species when in contact with water is reduced compared to the same non-passivated material.

“Keratinous materials” or “keratinous substance” means nails (finger and/or toe nails), skin such as body, face, and eye area, scalp, keratin fibers such as eyelashes, eyebrows, and hair, and mucous membranes such as lips.

“Physiologically acceptable” means compatible with keratinous materials and having a pleasant color, odor and feel, and which does not cause any unacceptable discomfort (stinging or tautness) liable to discourage a consumer from using the composition.

“UV protection efficiency” or “filtering efficiency” in the context of the present disclosure, is evaluated from one or more of SPF, UVAPF, Critical Wavelength, and UVA-I/UV ratio.

“SPF” (Sun Protection Factor) measures the level of protection against erythema provided by a composition. The SPF value corresponds to the ratio between the minimum erythemal dosage (MED) measured wearing the composition and the MED measured with bare skin. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989).

The evaluation of the SPF (Sun Protection Factor) can be performed, for example, in vitro with spectrophotometer by Labsphere (North Sutton, NH, USA). In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the Sun Protection Factor (SPF) can also be performed in-vivo according to the ISO 24444:2019 protocol “Cosmetics-Sun protection test methods-In-vivo determination of the sun protection factor (SPF).” Also, it can be determined according to FDA protocols, as described in the document “Labeling and Effectiveness Testing; Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

The evaluation of UVA protection can also be measured in vitro with the Labsphere® spectrophotometer under conditions such as those discussed above in connection with SPF. ISO 24443:2021 protocol describes such an in vitro method.

FDA broad spectrum testing procedures, in particular “critical wavelength” testing procedures, can also be found at 21 C.F.R. Part 352 Subpart D § 352.72. Also, broad spectrum testing procedures include determining the UVA1/UV ratio as described in “Sunscreen Drug Products for Over-the-Counter Human Use” published in the Federal Register https://www.federalregister.gov/documents/2019/02/26/2019-03019/sunscreen-drug-products-for-over-the-counter-human-use.

According to the present disclosure, compositions of the present disclosure preferably have one or more of the following properties:
Compositions have a critical wavelength as determined by FDA's critical wavelength procedures of at least 370 nm;
Compositions have an SPF value of at least 15, preferably at least 30, preferably at least 50 and preferably at least 70;
Compositions have a UVAPF/SPF ratio of at least 1/3, and preferably at least 2/5; and/or
Compositions have a UVA1/UV ratio of 0.7 or higher, preferably 0.75 or higher, and preferably 0.8 or higher.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratinous material such as skin and evaluating the color of the composition after an extended period of time. For example, the color of a composition may be evaluated immediately following application to keratinous material such as skin and these characteristics may then be re-evaluated and compared after a certain amount of time. Further, these characteristics may be evaluated with respect to other compositions, such as commercially available compositions.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means about 20-25° C.

“Atmospheric pressure” means about 760 mmHg, i.e., about 105 pascals.

“UV filter” and “sunscreen agent” are used interchangeably in this application.

“UV efficacy” and “UV efficiency” and “UV protection efficacy” are used interchangeably in this application.

The compositions and methods of the present disclosure can comprise, consist of, or consist essentially of the essential elements and limitations of the disclosure described herein, as well as any additional or optional ingredients, components, or limitations described herein or otherwise useful. For example, the UV (ultraviolet) absorbing system of the compositions of the disclosure can “consist essentially of” at least one physical ultraviolet (UV)-attenuating material(s).

For purposes of the present disclosure, the “basic and novel property” associated with compositions, components and methods related to UV protection properties which “consist essentially of” identified ingredients or actions is “UV protection efficiency.”

For purposes of the present disclosure, the “basic and novel property” associated with compositions, components and methods related to composition properties prior to application to keratinous material “consisting essentially of” identified ingredients or actions is “stability against phase separation.”

Compositions of the present disclosure may be in any form suitable for use as a personal care composition, such as that of a stick, a paste, a cream, an anhydrous composition, an emulsion (oil-in-water, water-in-oil, multiple emulsion such as oil-in-water-in-oil), nanoemulsion, a gel, a liquid, a solid, etc. These compositions can be used for any personal care purpose in cosmetic and/or dermatological products such as, for example, a sunscreen, a foundation, lip balms, lipsticks, concealers, mascaras, leave-in hair products, eye shadows, powders, etc.

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Composition

### Physical UV-Attenuating Material

According to the present disclosure, compositions comprising at least one physical ultraviolet (UV)-attenuating material are provided. “Physical ultraviolet (UV)-attenuating material” as used herein refers to solid inorganic ingredients which absorb incoming ultraviolet (UV)-light, and may optionally scatter incoming ultraviolet (UV)-light, when present in compositions disclosed herein. The physical UV-attenuating material preferably comprises metal oxide(s) such as, for example, oxides of titanium, chromium, zinc, tin, alumina, cerium, and/or iron. Specific examples of suitable metal oxide(s) include, but are not limited to, at least one metal oxide selected from the group consisting of titanium dioxide, zinc oxide, iron oxide, chromium oxide, tin oxide, alumina, cerium oxide, and mixtures thereof.

The physical UV-attenuating material may be subjected to a surface treatment agent to improve sensory, performance, and/or compatibility of the compositions disclosed herein. Suitable surface treatment agents may include hydrophobic or hydrophilic surface treatment agents such as, for example, those described in Cosmetics & Toiletries, February 1990, Vol. 105, pp. 53-64, including but not limited to specific examples such as amino acids, beeswax, fatty acids, fatty acid salts, fatty alcohols, anionic surfactants, lecithin, lecithin derivatives, metal alkoxides, polyethylene, silicones, proteins, alkanolamines, silicon oxides, metal oxides, sodium hexametaphosphate, alumina, and/or glycerol. Preferably, the physical UV-attenuating material is passivated.

Preferably, the physical UV-attenuating material comprises one or more of zinc oxide and/or titanium dioxide. Preferably, the physical UV-attenuating material comprising one or more of zinc oxide and/or titanium dioxide is passivated.

According to preferred embodiments, the physical UV-attenuating material comprises titanium dioxide. Titanium dioxide may be present in any form in the compositions disclosed herein. Further, the TiO2 can be treated (coated) or untreated.

According to preferred embodiments, the physical UV-attenuating material comprises zinc oxide. Zinc oxide may be present in any form (e.g., wurtzite or zincblende form) in the compositions disclosed herein. Further, the zinc oxide can be treated (coated) or untreated.

Preferably, the physical UV-attenuating material comprises zinc oxide. Preferably, the zinc oxide is passivated.

Preferably, the physical UV-attenuating material comprises titanium dioxide. Preferably, the titanium dioxide is passivated.

Preferably, mean primary particle size of the physical ultraviolet (UV)-attenuating material is from 1 nm to 500 nm, preferably from 5 nm to 250 nm, preferably from 10 nm to 100 nm, and preferably from 20 nm to 50 nm, including all ranges and subranges therebetween such as, for example, 25 nm to 40 nm, 10 nm to 75 nm, 10 nm to 40 nm, and 15 nm to 150 nm.

Suitable examples of coated pigments include but are not limited to titanium dioxides that have been coated such as titanium dioxides:
with hydrated silica, such as the product MT-100WP from Tayca,
with silica and iron oxide, such as the product Sunveil FR from Ikeda,
with silica and alumina, such as the products MT-500SA® and MT-100SA® from Tayca and Tioveil™ AQ-N from Croda,
with alumina, such as the product TTO-55 (A)® from Ishihara,
with alumina and aluminium stearate, such as the products MT-100TV®, MT-100Z® and MT-01® from Tayca, the product Solaveil™ CT100® from Croda and the product Eusolex T-AVO® from Merck,
with silica, alumina and alginic acid, such as the product MT-100AQ® from Tayca,
with alumina and aluminium laurate,
with iron oxide and iron stearate,
with zinc oxide and zinc stearate,
with silica and alumina and treated with a silicone, such as the products MTY-500SAS® or Microtitanium Dioxide MT-100SAS® from Tayca,
with silica, alumina, and aluminium stearate and treated with a silicone,
with silica and treated with a silicone,
with silica and treated with a silicone, such as the product TTO-55 (S)® from Ishihara,
with triethanolamine,
with stearic acid, such as the product TTO-55 (C)® from Ishihara,
with sodium hexametaphosphate,
with octyltrimethylsilane,
with a polydimethylsiloxane, with anatase/rutile TiO2 treated with a polydimethylhydrogenosiloxane,
with triethylhexanoin, with aluminium stearate and with alumina sold under the trade name Solaveil™ CT-200 by Croda,
with aluminium stearate, with alumina and with silicone, sold under the trade name Solaveil™ CT-12W by Croda,
with lauroyl lysine, and/or
with C₉-C₁₅ fluoroalcohol phosphate and with aluminium hydroxide.

Mention may also be made of TiO₂ pigments doped with at least one transition metal such as iron, zinc or manganese, preferably manganese. Preferably, the doped pigments are in the form of an oily dispersion. The oil present in the oily dispersion is preferably chosen from triglycerides such as, for example, capric/caprylic acids. The oily dispersion of titanium oxide particles may also comprise one or more dispersants, for instance a sorbitan ester, for instance sorbitan isostearate, or a polyoxyalkylenated fatty acid ester of glycerol, for instance Tri-PPG-3 myristyl ether citrate and polyglyceryl-3 polyricinoleate. Preferably, the oily dispersion of titanium oxide particles includes at least one dispersant chosen from polyoxyalkylenated fatty acid esters of glycerol. Mention may be made more particularly of the oily dispersion of TiO₂ particles doped with manganese in capric/caprylic acid triglyceride in the presence of Tri-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold under the trade name Optisol™ OTP-1 by Croda.

Suitable uncoated titanium oxide includes, but it not limited to, those sold by Tayca under the trade names MT-500B or MT-600BR, or by Evonik under the name Degussa P 25.

Suitable examples of uncoated zinc oxide include, but are not limited to, zinc oxide marketed under the name “Z-COTE”® by BASF, zinc oxide marketed under the name “NanoArc® Zinc Oxide” by the company Nanophase Technologies, zinc oxide marketed under the name “MZ-500”, “MZ-300”, “MZ-200” or “MZ-150” by TAYCA.

Treated (coated) zinc oxide compounds are compounds that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum), polyethylene, silicones, hydrated silica, proteins (collagen, elastin), alkanolamines, silicon oxides, triethoxycaprylylsilane, metal oxides or sodium hexametaphosphate.

Examples of suitable coated zinc oxide include, but are not limited to, zinc oxide coated with polymethylhydrogensiloxane; zinc oxide dispersed in C12-15 alkyl benzonate (INCI: Zinc Oxide (and) C12-15 Alkyl Benzoate (and) Polyhydroxystearic Acid (and) Isostearic Acid), marketed by Croda under the tradename Sovaveil CZ-100; zinc oxide dispersions in C9-12 alkane with a dispersing agent, marketed under the tradename “DAITOPERSION Zn-60VA”® by the company Daito Kasei; ZnO coated with silicone grafted acrylic polymer, dispersed in cyclodimethylsiloxane, marketed under the name “SPD-Z5®” by Shin-Etsu; ZnO coated with hydrated silica, marketed by TAYCA under the name “MZ-500HP”; ZnO coated with hydrated silica, triethoxysilylethyl polydimethylsiloxyethyl hexyl dimethicone and hydrogen dimethicone (H-Me-Si), marketed by TAYCA under the name MZ-510 HPSX; ZnO coated with stearic acid or isostearic acid, such as those marketed by TAYCA under the name “MZ-505T”, “MZY-505EX” or “MZY-304EX”; ZnO coated with silicone oil, such as those marketed by TAYCA under the name “MZX-510HPS”, “MZY-505S”, “MZY-510M3S”, “MZ-505M”, “MZY-303S”, “MZY-303M”, “MZY-203S”, “MZY-210M3S” or “MZY-153S”; ZnO coated with triethoxycaprylylsilane, such as those sold by BASF under the name Z-COTE HP1, or by TAYCA under the name “MZX-508OTS”, “MZY-203OTS” or “MZX-304OTS” or by DSM under the name PARSOL ZX; for example: ZnO marketed under the trademark “Oxide Zinc CS-5” by Toshiba (ZnO coated with polymethylhydrosiloxane); ZnO marketed under the trademark “Nanogard Zinc Oxide FN” by Nanophase Technologies (as a 40% dispersion in Finsolv TN, C12-C15 alkyl benzoate); ZnO marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); ZnO marketed under the trademark “NFD Ultrafine ZnO” by Daikin (ZnO coated with phosphate of perfluoroalkyl and a copolymer based on perfluoroalkylethyl as a dispersion in cyclopentasiloxane); ZnO marketed under the trademark “SPD-Z1” by Shin-Etsu (ZnO coated with a silicone-grafted acrylic polymer dispersed in cyclodimethylsiloxane); ZnO marketed under the trademark “Escalol Z100” by ISP (alumina-treated ZnO dispersed in an ethylhexyl methoxycinnamate/PVP-hexadecene copolymer/methicone mixture); ZnO marketed under the trademark “Fuji ZnO-SMS-10” by Fuji Pigment (ZnO coated with silica and polymethylsilsesquioxane); and ZnO marketed under the trademark “Nanox Gel TN” by Elementis (ZnO dispersed at 55% in C12-C15 alkyl benzoate with hydroxystearic acid polycondensate); ZnO marketed under the trademark Finex by SAKAI such as FINEX-50LP, FINEX-50S-LP2 and FINEX-30S-LPT (ZnO coated with Hydrogen dimethicone); FINEX-33W (ZnO coated with Hydrated silica), FINEX-52W-LP2 and FINEX-33W-LP2 (ZnO coated with Hydrogen dimethicone and Hydrated silica), FINEX-50-OTS and FINEX-30-OTS (ZnO coated with Triethoxycaprylysilane).

Preferred coatings for zinc oxide preferably comprise one or more of the following: hydrated silica, triethoxysilylethyl polydimethylsiloxyethyl, hexyl dimethicone, hydrogen dimethicone, and/or triethoxycaprylysilane.

According to preferred embodiments of the present disclosure, the zinc oxide may be in platelet form, and may be coated or uncoated. Suitable examples of such forms are sold by Croda under the Solaveil (MicNo) name such as Solaveil MXP3, MZP7, MZP8, MZ3-100, MZ3-300 AND MZ7-100. Preferably, zinc oxide platelets useful according to the present disclosure (1) have a median specific surface area of more than 25 square meters per gram, preferably greater than 30 square meters per gram, and/or (2) are transparent (that is, >30% transmission at 600 nm). Suitable examples of such platelet forms can also be found in U.S. Pat. No. 11,608,275, the entire contents of which is hereby incorporated by reference in its entirety.

Suitable examples of other coated oxides include, but are not limited to, coated oxides preferably having amphiphilic properties such as:
titanium oxides coated with cetyl phosphate and with silica, such as the product “Eusolex® T-EASY” from Merck;
titanium oxides coated with polyglyceryl-10 oleate and with stearic acid, such as “MTY-200STW” from Tayca;
zinc oxides coated with polyglyceryl-10 oleate and with isostearic acid, such as “MZY-505EXW” from Tayca;
titanium oxides coated with polysorbate 80 and with isostearic acid, such as “MT-10EXW” from Tayca; and
zinc oxides coated with polysorbate 80 and with isostearic acid, such as “MZY-304EXW” from Tayca.

Preferably, the at least one physical ultraviolet (UV)-attenuating material is/are present in compositions of the present disclosure in an amount of at least about 5% by weight, preferably at least about 10% by weight, preferably at least about 12% by weight, preferably at least about 14% by weight, and preferably at least about 15% by weight, with the upper end of the range of the at least one physical ultraviolet (UV)-attenuating material present preferably being about 40% by weight (e.g., about 5-40%, about 10-40%, about 12-40%, etc.), preferably about 30% by weight (e.g., about 5-30%, about 10-30%, about 12-30%, etc.), preferably about 25% by weight (e.g., about 5-25%, about 10-25%, about 12-25%, etc.), and preferably about 20% by weight (e.g., about 5-20%, about 10-20%, about 12-20%, etc.), with all weights being based on the total weight of the composition.

According to preferred embodiments, compositions of the present disclosure contain a UV absorbing system essentially containing physical ultraviolet (UV)-attenuating material(s) as defined above.

### Additional Sunscreen Agents

According to preferred embodiments of the present disclosure, compositions optionally further comprising at least one additional UV filter (in addition to at least one physical ultraviolet (UV)-attenuating material) selected from the group consisting of organic UV filters are provided. However, as noted above, preferred embodiments of the present disclosure include compositions of the present disclosure containing a UV absorbing system containing little or no organic UV filter as defined above.

Additional organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Suitable organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds such as Bis-Ethylhexyloxyphenol Methoxyphenyl Triazine; benzotriazole compounds; benzalmalonate compounds including those mentioned in U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from -alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. Preferably, the lipophilic organic UV filters are selected from salicylic compounds, dibenzoylmethane compounds, benzylidene camphor compounds; benzophenone compounds; triazine compounds; benzotriazole compounds; as well as other categories of compounds identified herein; and mixtures thereof.

Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β,β-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Heliopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Heliopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxybenzoyl)benzoate (such as that marketed under the tradename UVINUL A+ by BASF).

Examples of triazine compounds include 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine (BEMT)), Diethylhexyl butamido triazone, such as that marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzalmalonate compounds include Dineopentyl 4′-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669,323 and U.S. Pat. No. 2,463,264.

Suitable para-aminobenzoic acid compounds include PABA (p-aminobenzoic acid), ethyl PABA, Ethyl dihydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis [6-(2H-benzotriazol-2-yl)-4-methyl-phenol], such as that marketed under the trademark “Mixxim BB/200” by Fairmount Chemical, 2,2′-methylenebis [6-(2H-benzotriazol-2-yl)-4-(1,1,3,3-tetramethylbutyl) phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or-“Mexoryl XL” by L'Oréal.

Examples of benzoxazole compounds include 2,4-bis [5-1(dimethylpropyl)benzoxazol-2-yl-(4-phenyl)imino]-6-(2-ethylhexyl)imino-1,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include 1,1-dicarboxy (2,2′-dimethylpropyl)-4,4-diphenylbutadiene.

If present, the at least one additional organic UV filter is preferably present in compositions of the present disclosure in an amount of at least about 1% by weight, preferably at least about 5% by weight, preferably at least about 10% by weight, preferably at least about 12.5% by weight, and preferably at least about 15% by weight, with the upper end of the range of additional UV filter present preferably being about 40% by weight (e.g., about 1-40%, about 10-40%, about 12.5-40%, etc.), preferably about 30% by weight (e.g., about 5-30%, about 10-30%, about 15-30%, etc.), preferably about 25% by weight (e.g., about 5-25%, about 10-25%, about 15-25%, etc.), and preferably about 20% by weight (e.g., about 1-20%, about 5-20%, about 10-20%, etc.), with all weights being based on the total weight of the composition.

According to preferred embodiments, compositions of the present disclosure comprise 10% or less by weight relative to the total weight of composition of such optional additional UV filters, preferably less than 7.5% by weight relative to the total weight of composition, preferably less than 5% by weight relative to the total weight of composition, preferably less than 3% by weight relative to the total weight of composition, and preferably less than 1% by weight relative to the total weight of composition.

According to preferred embodiments, compositions of the present disclosure further comprise at least one additional organic UV filter selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, Octocrylene, and mixtures thereof. In such embodiments, the UV absorbing system can “consist of” or “consist essentially of” (1) at least one physical ultraviolet (UV)-attenuating material and (2) at least one organic UV filter selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, Octocrylene, and mixtures thereof.

According to other preferred embodiments, however, compositions of the present disclosure are “free of,” “substantially free of,” or “devoid of” as defined above of one or more of additional organic UV filters selected from the group consisting of 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), Avobenzone (butyl methoxydibenzoylmethane), Octisalate (ethylhexyl salicylate), Ensulizole (Phenylbenzimidazole sulfonic acid), Homosalate, and Octocrylene, preferably two or more, preferably three or more, preferably four or more, or preferably all five of these sunscreen agents.

According to preferred embodiments, compositions of the present disclosure are “free of,” “substantially free of,” or “devoid of” as defined above of one or more of additional organic UV filters selected from the group consisting of OXYBENZONE (benzophenone-3), OCTINOXATE (Ethylhexyl methoxycinnamate), ETHYLHEXYL TRIAZONE, DROMETRIZOLE TRISILOXANE, METHYLENE BIS-BENZOTRIAZOLYL TETRAMETHYLBUTYL PHENOL, DIETHYLAMINO HYDROXY BENZOYL HEXYL BENZOATE, DIETHYLHEXYL BUTAMIDO TRIAZONE, ISOAMYL P-METHOXYCINNAMATE, POLYSILICONE-15, 4-METHYLBENZYLIDENE CAMPHOR, DISODIUM PHENYL DIBENZIMIDAZOLE TETRASULFONATE, METHOXYPROPYLAMINO CYCLOHEXENYLIDENE ETHOXYETHYLCYANOACETATE, 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine), preferably two or more, preferably three or more, preferably four or more, etc., and preferably “free of,” “substantially free of,” or “devoid of” all of these sunscreen agents.

According to preferred embodiments, compositions of the present disclosure are “free of,” “substantially free of,” or “devoid of” as defined above of OXYBENZONE (benzophenone-3) and/or OCTINOXATE (Ethylhexyl methoxycinnamate) and/or 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine).

According to preferred embodiments, the UV absorbing system of compositions of the present disclosure can “consist of” or “consist essentially of” at least one physical ultraviolet (UV)-attenuating material.

According to preferred embodiments, this disclosure contemplates omitting one or more of any of the specific UV filters discussed above from the UV absorbing system of compositions of the present disclosure. By way of example, octocrylene and/or octinoxate can be omitted from the compositions. Similar omission of one or more of any of the specific UV filters discussed is thus contemplated.

### Surfactant Component (System)

According to present disclosure, compositions comprising a surfactant component are provided. Preferably, the compositions are emulsion compositions. Generally speaking, acceptable surfactants (emulsifiers) can be chosen from ionic emulsifiers, nonionic emulsifiers, and mixtures thereof.

According to preferred embodiments of the present disclosure, the surfactant component comprises at least one fatty acid ester of polyol. “Fatty acid ester of polyol” as used herein means an ester of a fatty acid (or fatty acid polymer) and of polyol in which the fatty acid comprises a C₆-C₂₂, preferably C₁₆-C₂₀, alkyl chain and the polyol is chosen from glycerol, polyglycerol, sorbitan, and mixtures thereof. The fatty acid may also be in a polymeric form, as is the case for polyhydroxystearic acid (12-hydroxystearic acid polymer).

Examples of linear- or branched-chain C₁₆-C₂₀ fatty acids include, but are not limited to, stearic acid, isostearic acid, lauric acid, myristic acid, palmitic acid. Examples of unsaturated chain C₁₆-C₂₀ fatty acids include, but are not limited to, oleic acid and linoleic acid. An example of a C₁₆-C₂₀ fatty acid polymer that may be mentioned is poly(12-hydroxystearic acid).

Polyglycerols are compounds having formula:

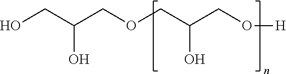

in which the degree of condensation n ranges from 1 to 11, preferably from 2 to 6 and preferably from 3 to 6.

According to preferred embodiments, the ester of a fatty acid and a polyol contains 2 to 10 moles (units) of polyols, preferably 2 to 4 moles of polyols, in particular 2 to 4 units of glycerol or a mixture of polyglycerols (glycerol, di-, tri-, tetra-, penta-, oligoglycerols). Preferably, the ester of a fatty acid and a polyol contains 4 mol (or units) of polyol, in particular 4 mol (or units) of glycerol.

According to preferred embodiments, the ester of a fatty acid and a polyol comprises a fatty acid ester of a dicarboxylic acid having from 2 to 16 carbon atoms, preferably from 8 to 14 carbon atoms, such as azelaic acid, sebacic acid, and dodecanedioic acid, and a polyol.

Suitable examples of esters of fatty acids and polyols include, but are not limited to, esters of isostearic acid and polyols and mixtures thereof, for example esters of isostearic acid and glycerol and/or of sorbitan, such as for example polyglycerolated (4 moles) isostearate (INCI name: Polyglyceryl-4 Isostearate) sold under the name Isolan GI34® by Goldschmidt, polyglycerolated (3 mol) diisostearate sold under the name Lameform TGI® by the company Cognis; polyglycerolated distearate (2 mol) sold under the name Emalex PGSA® by the company Nihon emulsion; polyglycerolated (10 mol) monoisostearate sold under the name Nikkol Decaglyn 1-IS® by the company Nihon Surfactant (INCI name: Polyglyceryl-10 isostearate); polyglyceryl-4 diisostearate polyhydroxystearate sebacate sold under the name Isolan GPS® by the company Goldschmidt; the mixture of sorbitan isostearate and glyceryl isostearate, such as the product sold under the name Arlacel 986® by the company ICI, the mixture of sorbitan isostearate and polyglyceryl (3 mol) isostearate sold under the name Arlacel 1690® by the company Unigema, and mixtures thereof.

According to preferred embodiments, fatty acid esters of polyglycerol include, but are not limited to, polyglycerolated (4 mol) I isostearate (INCI name: Polyglyceryl-4 isostearate) sold under the name Isolan GI34® by Goldschmidt, polyglyceryl-4 diisostearate polyhydroxystearate sebacate sold under the name Isolan GPS® by Goldschmidt, the mixture of sorbitan isostearate and polyglyceryl (3 mol) isostearate sold under the name Arlacel 1690® by the company Unigema, and mixtures thereof.

According to preferred embodiments, the fatty acid ester of a polyol is an ester of poly(12-hydroxystearic acid) and of dicarboxylic acids obtained by esterification of a mixture of polyglycerol with (i) a polyhydroxystearic acid, with from 1 to 10, preferably from 2 to 8, preferably from 2 to 5 polyglycerol units (preferably 4 units), (ii) linear or branched aliphatic dicarboxylic acids containing from 2 to 16 carbon atoms, preferably from 4 to 14 carbon atoms (preferably sebacic acid), and (iii) saturated or unsaturated, linear or branched fatty acids containing from 6 to 22 carbon atoms, preferably from 16 to 20 carbon atoms (preferably isostearic acid).

Preferably, the degree of esterification of the polyglyceryl mixture is between 20% and 40%, preferably between 40% and 70%.

Suitable poly(12-hydroxystearic acid) esters of polyglyceryl are described in U.S. patent application 2005/0031580, the disclosure of which is hereby incorporated herein by reference.

According to one preferred embodiment, the fatty acid ester of a polyol is an ester of poly(12-hydroxystearic acid) and of dicarboxylic acids obtained by esterification of a mixture of polyglycerol with (i) a polyhydroxystearic acid, with from 2 to 5 polyglycerol units, (ii) linear or branched aliphatic dicarboxylic acids containing 4 to 14 carbon atoms, and (iii) saturated or unsaturated, linear or branched fatty acids containing from 16 to 20 carbon atoms. Preferably, the fatty acid ester of a polyol is an ester of polyhydroxystearic acid and of dicarboxylic acids obtained by esterification of a mixture of polyglycerol with (i) a polyhydroxystearic acid, with from 2 to 5 polyglycerol units (preferably 4 units), (ii) linear or branched aliphatic dicarboxylic acids containing 4 to 14 carbon atoms (preferably sebacic acid) and (iii) saturated or unsaturated, linear or branched fatty acids containing from 16 to 20 carbon atoms (preferably isostearic acid). Preferably, the fatty acid ester of a polyol is polyglyceryl-4 diisostearate polyhydroxystearate sebacate of formula:

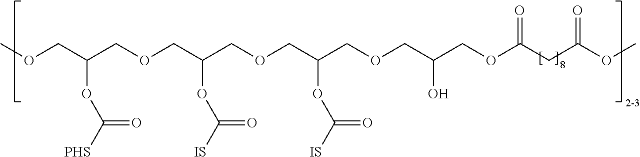

in which PHS denotes polyhydroxystearic acid and IS denotes isostearic acid and INCI name: Polyglyceryl-4 Diisostearate/Polyhydroxystearate/Sebacate. Such a compound can be prepared according to US 2005/0031580 and sold under the name Isolan GPS® by the company Evonik Goldschmidt (Degussa).

According to preferred embodiments, the polyglycerol ester with a poly(hydroxystearic) acid and carboxylic acids is used in mixture with other polyglycerol esters, in particular comprising 3 polyglycerol units, with at least one fatty acid, in particular an oleic acid, preferably in the mixture Polyglyceryl-4 Diisostearate/Polyhydroxystearate/Sebacate (and) Caprylic/Capric Triglyceride (and) Polyglyceryl-3 Oleate (and) Diisostearoyl Polyglyceryl-3 Dimer Dilinoleate sold under the name ISOLAN® 17 MB by the company EVONIK GOLDSCHMIDT.

According to preferred embodiments, the surfactant component additionally or alternatively comprises at least one fatty acid ester of polyol which is a C₁₆-C₂₀ fatty acid ester of sorbitan, such as, for example, sorbitan stearate, sorbitan isostearate, and/or sorbitan oleate,

Preferably, the surfactant(s) (surfactant component) is/are present in the compositions of the present disclosure in an amount of from about 1% to about 20%, preferably from about 2% to about 17.5%, preferably from about 3% to about 15%, and preferably from about 4% to about 12%, by weight based on the total weight of the composition, including all ranges and subranges in between.

### Oil

According to preferred embodiments of the present disclosure, compositions further comprising at least one oil are provided. “Oil” means a substance which is hydrophobic and lipophilic, and is a liquid at about room temperature (20 to 25° C.) and about atmospheric pressure (760 mm Hg).

Suitable oils include volatile and/or non-volatile oils. Such oils can be any acceptable oil including but not limited to silicone oils and/or hydrocarbon oils.

According to certain embodiments, the compositions of the present disclosure preferably comprise one or more volatile silicone oils. Examples of such volatile silicone oils include linear or cyclic silicone oils having from 2 to 7 silicon atoms, these silicones being optionally substituted with alkyl or alkoxy groups of 1 to 10 carbon atoms. Specific oils that may be used in the disclosure include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC | 56 | 1.5 |
| 200 (1.5 cSt) from Dow Corning | | |
| PDMS DC 200 (2 cSt) from | 87 | 2 |
| Dow Corning | | |

Further, a volatile linear silicone oil may be employed in the present disclosure. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present disclosure, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol | 60 |
| n-butyl ether | |
| Ethyl 3-ethoxypropionate | 58 |
| Propylene glycol | 46 |
| methylether acetate | |
| Isopar L (isoparaffin C₁₁-C₁₃) | 62 |
| Isopar H (isoparaffin C₁₁-C₁₂) | 56 |

According to certain embodiments of the present disclosure, the composition comprises at least one non-volatile oil. Examples of non-volatile oils that may be used in the present disclosure include, but are not limited to, polar oils such as, for example:
esters and ethers, in particular fatty acids, such as oils of formulas R1COOR2, wherein R1 represents the remainder of a fatty acid having from 8 to 29 carbon atoms, and R 2 represents a hydrocarbon chain, branched or not, containing from 2 to 30 carbon atoms, such as for example Purcellin oil, ethyl oleate, ethyl stearate, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, fatty alcohol decanoates; polyol esters, such as propylene glycol dioctanoate, neopentylglycol diheptanoate and diethylene glycol diisononanoate; glyceryl triesters such as triesters of glycerol and of C12-C22 fatty acid, preferably C16-C18 fatty acid, preferably C18 fatty acid, such as triester of glycerin and isostearic acid (triisostearin), triester of glycerin and stearic acid (tristearin), triester of glycerin and oleic acid (triolein), glyceryl tripalmitate, glyceryl trilaurate, etc.; and esters of pentaerythritol such as pentaerythrityl tetraisostearate or dipentaerythrityl pentaisononanoate;
ethers containing from 10 to 40 carbon atoms;
liquid C₈ to C₂₆ fatty alcohols, for instance oleyl alcohol, cetyl alcohol, stearyl alcohol, octyldodecanol, and cetearly alcohol;
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or, for example, sunflower, corn, soybean, squash, grape seed, sesame, hazelnut, apricot, macadamia, ara, sunflower oil, castor oil, avocado, triglycerides of caprylic/capric acids such as those sold by the company Stearineries Dubois or those sold under the names Miglyol 810, 812 and 818 by the company Dynamit Nobel, coco-caprylate/caprate (esterified oil from coconut oil), jojoba oil, shea butter oil; and
mixtures thereof.

Further, examples of non-volatile oils that may be used in the present disclosure include, but are not limited to, non-polar oils such as branched and unbranched hydrocarbons, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present disclosure, the compositions of the present disclosure comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Particularly preferred oils include, but are not limited to, one or more of the following: Diisopropyl Sebacate, C12-15 Alkyl Benzoate, Phenethyl Benzoate, Isopropyl Lauroyl Sarcosinate, Diisopropyl Adipate, Dibutyl Adipate, Dicaprylyl Carbonate, Dicaprylate/Dicaprate, Coco Glycerides, Caprylic/capric triglyceride, Isopropyl Myristat, Isopropyl Palmitate, Coco Caprylate/Caprate, Ethylhexyl Palmitate, Isononyl isononanoate, Octyl dodecanol, Isohexadecane, isododecane, Dicaprylyl Ether, C15-19 Alkane, and mixtures thereof.

According to preferred embodiments, the at least one oil is/are present in the compositions of the present disclosure in an amount ranging from about 1% to about 70% by weight, more preferably from about 5 to about 60% by weight, and preferably from about 10% to about 50% by weight, based on the total weight of the composition, including all ranges and subranges within these ranges such as, for example, 15% to 40%, 20% to 45%, etc.

### Aqueous Phase

The compositions of the present disclosure may also optionally contain water. When the compositions of the present disclosure contain water, they are preferably in the form of an emulsion. Preferably, when the compositions of the present disclosure contain water, they are in the form of an emulsion containing an external aqueous phase such as an oil-in-water emulsion (O/W) or a water-in-oil-in-water emulsion (O/W/O), or an emulsion containing an external oil phase such as a water-in-oil emulsion (W/O) or an oil-in-water-in-oil (O/W/O) emulsion. Preferably, when in the form of an emulsion, the oil phase can contain silicone oils (e.g., Si/W or W/Si emulsion) or hydrocarbon oils. When present, water is preferably present in an amount of from about 10% to about 80% by weight, preferably from about 15% to about 60% by weight, preferably from about 20% to about 40% by weight, including all ranges and subranges therebetween, all weights being based on the total weight of the composition.

According to preferred embodiments, however, compositions of the present disclosure are devoid of water, substantially free of water, or free of water as defined herein. Preferably, the compositions of the present disclosure are anhydrous.

If present in compositions of the present disclosure, the aqueous phase may comprise at least one water-soluble organic solvent which is liquid at room temperature and atmospheric pressure. For example, such at least one water-soluble organic solvent may include:
C1-C5 monoalcohols having a C1-C5 alkane chain and a single hydroxyl function (OH). Suitable C1-C5 monoalcohols include methanol, ethanol, propanol, isopropanol, butanol and mixtures thereof;
polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;
and mixtures thereof.

According to preferred embodiments, the at least one water-soluble organic solvent is selected from the group consisting of ethanol, dipropylene glycol, butylene glycol, propanediol and propylene glycol, and mixtures thereof.

If present, the water-soluble organic solvent(s) is/are preferably present in compositions of the present disclosure in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

### Additional Optional Ingredients

Compositions of the present disclosure may also optionally further include at least one additive or auxiliary commonly used in cosmetic compositions and known to a person skilled in the art as being capable of being incorporated into such compositions. Such additives or auxiliaries may be chosen from film formers, coloring agents (e.g., dyes and pigments), waxes, thixotropic agents (e.g., clays), fillers, preservatives, fragrances, antioxidants, agents for combating free radicals, spreading agents, dispersing agents, antifoaming agents, neutralizing agents, stabilizing agents, active principles chosen from essential oils, moisturizing agents, vitamins, actives, proteins, ceramides, plant extracts, fibers, and the like, wetting agents and their mixtures. Although, preferably, compositions of the present disclosure are “free of,” “substantially free of,” or “devoid of” such additives.

A person skilled in the art will take care to select the optional additional additives and/or the amount thereof such that the advantageous properties of the composition according to the disclosure are not, or are not substantially, adversely affected by the envisaged addition.

Needless to say, the composition of the disclosure should be cosmetically or dermatologically acceptable, i.e., it should contain a non-toxic physiologically acceptable. The composition may be in any galenic form normally employed in the cosmetic and dermatological fields which is suitable for topical administration as discussed above.

These auxiliary additives may be present in the composition in a proportion from 0% to 99% (such as from 0.01% to 90%) relative to the total weight of the composition and further such as from 0.1% to 50% (if present), including all ranges and subranges therebetween.

In accordance with the present disclosure, compositions of the present disclosure can be a stand-alone product (for use by itself), or they can be a product for use in conjunction with another composition, for example it can be a basecoat (primer) composition, a color coat composition, or a topcoat (over coat) composition. It should be understood that when compositions of the present disclosure are applied to keratinous materials in the form of any of such compositions, such application can comprise one or more layers of the product. Thus, for example, application of at least one color coat composition can comprise one or more color coat layers; application of the at least topcoat composition can comprise one or more topcoat layers; and application of the at least one basecoat composition can comprise one or more basecoat layers. Preferably, such basecoat, color coat and topcoat compositions contain three or fewer layers of compositions, preferably two or fewer layers of compositions, and preferably a single layer of compositions.

During application of compositions of the present disclosure, basecoat (if present) is typically applied directly to keratinous material, color coat is typically applied either directly to the keratinous material (if no basecoat is present) or to a previously applied basecoat, and topcoat (if present) is typically applied to a color coat.

According to preferred embodiments of the present disclosure, methods of treating, protecting, enhancing the appearance of, caring for and/or making up keratinous material by applying compositions of the present disclosure to the keratinous material in an amount sufficient to treat, enhance the appearance of, care for and/or make up the keratinous material are provided.

Preferably, “making up” keratinous material includes applying a composition comprising at least one coloring agent to the keratinous material in an amount sufficient to provide color and/or optical effect to the keratinous material.

Preferably, “protecting” keratinous material includes applying a composition of the present disclosure to protect keratinous material from damage resulting from exposure to UV rays.

In accordance with the preceding embodiments, compositions of the present disclosure are applied topically to the keratinous material in an amount sufficient to treat, enhance the appearance of, care for and/or make up the keratinous material. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

According to preferred embodiments of the present disclosure, methods of making compositions comprising at least one physical ultraviolet (UV)-attenuating material by combining at least one physical ultraviolet (UV)-attenuating material and at least one fatty acid ester of polyol during formation of the compositions are provided. Preferably, the compositions are fluid. Preferably, the compositions provide broad spectrum protection against UV rays. Preferably, the compositions provide SPF protection of at least 25, preferably at least 30. Preferably, the compositions are in the form of an emulsion, most preferably a water-in-oil emulsion.

The present disclosure also envisages kits and/or prepackaged materials suitable for consumer use containing one or more compositions according to the description herein, alone or in combination with other consumer care products such as makeup products such as basecoats, topcoats, removal compositions, etc. The packaging and application device for any subject of the disclosure may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result. The percentages are given on a weight basis.

### Example 1

The following compositions were prepared at room temperature (1 kg total quantity) with a Rayneri VMI rotor-stator tool. The mineral filter ZnO (C) was added slowly into the oil phase (A) (previously mixed with the dispersing agent (B)) under high shear (rotor stator 3000 rpm) at room temperature for 10 min. The emulsifier (D) was added and mixed for an additional 5 min. The water phase (E) (previously homogenized at room temperature for 10 min under medium shear) was added slowly for emulsification at medium shear (rotor stator 2500 rpm) for 10 min.

The stability of the prepared compositions was determined by visual observation at 24 hours, and the viscosity of the prepared compositions was measured at room temperature with a rotating viscometer LAMY RM180 equipped with a mobile Ms R 2 just after manufacturing (t0). The viscosity value was taken after 10 minutes of rotation of the tool in the cup to reach a plateau of the value, expressed in mPa·s by using a correspondence curve between raw data expressed in deviation units (UD), and mPa·s.

| Phase | INCI Name | Inv. 1 | Inv. 2 | Inv. 3 | Inv. 4 |
|---|---|---|---|---|---|
| A | ISOPROPYL MYRISTATE | 21.05 | 21.05 | 24.18 | 24.18 |
| A | ISOHEXADECANE | 3 | 3 | 3 | 3 |
| A | TRIISOSTEARIN | 8 | 8 | 8 | 8 |
| A | C15-19 ALKANE | 3 | 3 | 3 | 3 |
| A | ETHYL OLEATE | 5 | 5 | 5 | 5 |
| B | SORBITAN OLEATE | 2 | 0 | 2 | 0 |
| B | SORBITAN ISOSTEARATE | 0 | 2 | 0 | 2 |
| C | ZINC OXIDE | 15 | 15 | 15 | 15 |
| D | POLYGLYCERYL-4 | 5 | 5 | 0 | 0 |
| | DIISOSTEARATE/ | | | | |
| | POLYHYDROXYSTEARATE/ | | | | |
| | SEBACATE (and) | | | | |
| | CAPRYLIC/CAPRIC | | | | |
| | TRIGLYCERIDE (and) | | | | |
| | POLYGLYCERYL-3 OLEATE | | | | |
| | (and) DIISOSTEAROYL | | | | |
| | POLYGLYCERYL-3 DIMER | | | | |
| | DILINOLEATE | | | | |
| D | POLYGLYCERYL-4 | 0 | 0 | 1.88 | 1.88 |
| | DIISOSTEARATE/ | | | | |
| | POLYHYDROXYSTEARATE/ | | | | |
| | SEBACATE | | | | |
| E | MAGNESIUM SULFATE | 2 | 2 | 2 | 2 |
| E | PHENOXYETHANOL | 0.7 | 0.7 | 0.7 | 0.7 |
| E | WATER | 26 | 26 | 26 | 26 |
| E | ARGININE | 0 | 0 | 0 | 0 |
| E | GLYCERIN | 3 | 3 | 3 | 3 |
| E | SORBITOL | 3 | 3 | 3 | 3 |
| E | SODIUM CHLORIDE | 1 | 1 | 1 | 1 |
| E | TRISODIUM | 0.25 | 0.25 | 0.25 | 0.25 |
| | ETHYLENEDIAMINE | | | | |
| | DISUCCINATE | | | | |
| E | PENTYLENE GLYCOL | 2 | 2 | 2 | 2 |

| | Inv. 1 | Inv. 2 | Inv. 3 | Inv. 4 |
|---|---|---|---|---|
| Stability | Fully | Fully | Slight | Slight |
| at 24 hrs | stable, | stable, | exudation, | exudation, |
| (visual | homogeneous | homogeneous | thin clear | thin clear |
| observation) | | | phase on | phase on |
| | | | the top | the top |
| Viscosity | 183 | 216 | 107 | 107 |
| at t0 | | | | |
| (m · Pa · s) | | | | |

All compositions were stable and homogeneous after manufacturing, having the form of fluid white milks exhibiting low viscosity values. However, after 24 hours of storage, there was a slight difference. Compositions 1 and 2 (Inv. 1 and 2) were fully stable and homogeneous after 24 hours of storage, whereas compositions 3 and 4 (Inv. 3 and 4) were not, displaying a slight exudation with a thin clear phase on the top of the sample.

What is claimed is:

1. A composition comprising a UV absorbing system comprising at least one physical ultraviolet (UV)-attenuating material, preferably at least one metal oxide, preferably at least one metal oxide selected from oxides of titanium, chromium, zinc, tin, alumina, cerium, and/or iron, and a surfactant system comprising a surfactant system (component) comprising at least one fatty acid ester of polyol, wherein the composition is devoid of organic UV filters.

2. The composition of claim 1, wherein the at least one physical ultraviolet (UV)-attenuating material is present in the composition in an amount of at least 5% by weight with respect to the total weight of the composition, preferably about 5% to about 40% by weight with respect to the total weight of the composition.

3. The composition of claim 1, wherein the surfactant system comprises at least one fatty acid ester of polyol which is an ester of polyhydroxystearic acid and of dicarboxylic acids obtained by esterification of a mixture of polyglycerol with (i) a poly(hydroxystearic) acid, with from 2 to 5 polyglycerol units, (ii) linear or branched aliphatic dicarboxylic acids containing 4 to 14 carbon atoms and (iii) saturated or unsaturated, linear or branched fatty acids containing from 16 to 20 carbon atoms, preferably Polyglyceryl-4 Diisostearate/Polyhydroxystearate/Sebacate.

4. The composition of claim 3, wherein the polyglycerol with a poly(hydroxystearic) acid and dicarboxylic acids is used in mixture with at least one additional polyglycerol ester, preferably comprising 3 polyglycerol units, with at least one fatty acid, preferably oleic acid, preferably in the mixture with Polyglyceryl-4 Diisostearate/Polyhydroxystearate/Sebacate, Polyglyceryl-3 Oleate, and/or Diisostearoyl Polyglyceryl-3 Dimer Dilinoleate.

5. The composition of claim 1, wherein the surfactant system comprises at least one of sorbitan stearate, sorbitan isostearate, and sorbitan oleate.

6. The composition of claim 1, in the form of an emulsion, preferably a water-in-oil emulsion.

7. The composition of claim 1, wherein the composition is fluid.

8. The composition of claim 1, wherein the composition does not phase separate after 24 hours.

9. The composition of claim 1, wherein the at least one UV absorbing system essentially contains at least one physical ultraviolet (UV)-attenuating material.

10. The composition of claim 1, wherein the at least one physical ultraviolet (UV)-attenuating material is selected from the group consisting of titanium dioxide, zinc oxide, iron oxide, chromium oxide, tin oxide, alumina, cerium oxide, and mixtures thereof.

11. The composition of claim 1, wherein the at least one physical ultraviolet (UV)-attenuating material is surface treated with a surface treatment agent, preferably the surface treating agent comprising at least one of amino acids, beeswax, fatty acids, fatty acid salts, fatty alcohols, anionic surfactants, lecithin compounds, metal alkoxides, polyethylene, silicones, proteins, alkanolamines, silicon oxides, metal oxides different from the at least one physical ultraviolet (UV)-attenuating material, sodium hexametaphosphate, alumina, glycerol, and mixtures thereof.

12. The composition of claim 1, wherein the at least one physical ultraviolet (UV)-attenuating material comprises zinc oxide and/or titanium dioxide, preferably passivated.

13. The composition of claim 1, wherein the at least one physical ultraviolet (UV)-attenuating material comprises zinc oxide and/or titanium dioxide, preferably coated with a coating agent, preferably the coating agent comprising at least one of hydrated silica, triethoxysilylethyl polydimethylsiloxyethyl hexyl dimethicone, hydrogen dimethicone, triethoxycaprylylsilane, and mixtures thereof.

14. The composition of claim 1, wherein the at least one physical ultraviolet (UV)-attenuating material comprises zinc oxide, preferably passivated or coated with hydrated silica and/or hydrogen dimethicone.

15. The composition of claim 1, wherein the at least one physical ultraviolet (UV)-attenuating material has a mean primary particle size of from 1 nm to 500 nm, preferably from 5 nm to 250 nm, preferably from 10 nm to 100 nm, preferably from 20 nm to 50 nm.

16. The composition of claim 1, wherein the at least one physical ultraviolet (UV)-attenuating material has a mean primary particle size of about 10 nm to about 40 nm.
